(19) United States
(12) Patent Application Publication
(10) Pub. No.: US 2023/0218715 A1
(43) Pub. Date: Jul. 13, 2023

(54) COMPOSITIONS AND METHODS FOR PREVENTION OF CORONAVIRUS INFECTION

(71) Applicants: UNIVERSITY OF LOUISVILLE RESEARCH FOUNDATION, INC., Louisville, KY (US); UNIVERSITY OF PITTSBURGH - OF THE COMMONWEALTH SYSTEM OF HIGHER EDUCATION, Pittsburgh, PA (US)

(72) Inventors: Kenneth E. Palmer, Anchorage, KY (US); Joshua L. Fuqua, Peewee Valley, KY (US); Lisa Cencia Rohan, Pittsburgh, PA (US); Lin Wang, Pittsburgh, PA (US); Barry R. O'Keefe, Frederick, MD (US)

(21) Appl. No.: 17/999,235

(22) PCT Filed: May 18, 2021
(86) PCT No.: PCT/US21/33009

§ 371 Date: Nov. 18, 2022

Related U.S. Application Data
(60) Provisional application No. 63/026,375, filed on May 18, 2020.
(60) Provisional application No. 63/070,375, filed on Aug. 26, 2020.

(51) Int. Cl. A61K 38/16; A61K 9/00; A61K 47/36; A61P 31/14

(52) U.S. Cl. CPC A61K 38/168; A61K 9/0043; A61K 47/36; A61P 31/14

(57) ABSTRACT

The present invention relates to griffithsin polypeptides and methods of using the same in inhibition of viral infection. Certain embodiments of the present invention relate to modified griffithsin polypeptides and methods of inhibiting coronavirus infection in a host by administering modified griffithsin polypeptides to the upper respiratory tract of the host. Further embodiments relate to an intranasal spray formulation including griffithsin polypeptides in a composition including a preservative and a viscosity modifier.

### CROSS-REFERENCE TO RELATED APPLICATION

This application claims the benefit of U.S. provisional patent application Ser. No. 63/026,375 filed 18 May 2020 for COMPOSITIONS AND METHODS FOR PREVENTION OF CORONAVIRUS INFECTION and to U.S. provisional patent application Ser. No. 63/070,375 filed 26 Aug. 2020 for Q-GRIFFITHSIN NASAL SPRAY, both of which are incorporated herein by reference.

### FIELD OF THE INVENTION

The present invention relates to griffithsin polypeptides and methods of using the same in inhibition of viral infection. Certain embodiments of the present invention relate to modified griffithsin polypeptides and methods of inhibiting coronavirus infection in a host by administering modified griffithsin polypeptides to the upper respiratory tract of the host. Further embodiments relate to an intranasal spray formulation including griffithsin polypeptides in a composition including a preservative and a viscosity modifier.

### BACKGROUND OF THE INVENTION

The world is in the midst of a pandemic due to Coronavirus disease-19 (COVID-19) caused by severe acute respiratory syndrome Coronavirus 2 (SARS-CoV-2). There are no effective treatments or prophylactics against COVID-19. SARS-CoV-2 transmission occurs predominantly through oral and nasal routes leading to high viral replication in the upper respiratory tract—the nasopharynx and oropharynx—as well as the lung and gastrointestinal tissues. High viral replication in the nasopharynx in the early stages of infection, prior to symptom onset, accounts for the high transmissibility of SARS-CoV-2. Respiratory aerosols and droplets are likely the source of most human transmission events. Consequently, in the absence of effective personal protective equipment, a biomedical intervention that protects the upper airway from SARS-CoV-2 infection could have a major impact on limiting the epidemic.

Griffithsin—also referred to as GRFT—is a protein that was originally isolated from red algae. It binds the terminal mannose residues of N-linked glycans found on the surface of human immunodeficiency virus type 1 (HIV-1), HIV-2, and other enveloped viruses, including hepatitis C virus (HCV), severe acute respiratory syndrome coronavirus (SARS-CoV), various avian CoV subtypes, BCoV, IBV, MHV, PCoV, HCoV and mutants, JEV, SIV and SHIV. Its activity has also been demonstrated in Nipah, Ebola virus. Herpes, Influenza, and RSV. An engineered form of griffithsin (GRFT), Q-GRFT, has increased stability against oxidation and displays similar activity (see, U.S. Pat. No. 10,501,507). This increased stability is relevant to the design of a marketable pharmaceutical product.

Accordingly, compositions and methods for inhibition of SARS-CoV-2 infection in the upper respiratory tract, such as a Q-GRFT nasal spray and prophylactic use thereof, would be both highly desirable and beneficial.

### SUMMARY

The instant subject matter relates to relates to griffithsin polypeptides and methods of using the same in inhibition of viral infection. Certain embodiments of the present invention relate to modified griffithsin polypeptides and methods of using the same in inhibition of SARS-CoV-2 infection in the upper respiratory tract. Delivery of an effective dosage of griffithsin protein to the upper respiratory tract, such as via a nasal spray, may be used to inhibit infection by SARS-CoV-2, endemic coronaviruses, and other viruses and respiratory pathogens.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 is a chart depicting the equilibrium dissociation constant (KD) of Q-GRFT with various coronaviruses.

FIG. 2 is a pair of charts illustrating that GRFT treatment protects mice against morbidity from SARS-CoV infection. Mice were treated with sham control (no virus), GRFT alone, SARS-CoV alone, or GRFT followed with SARS-CoV infection. Animals were monitored daily for weight loss (panel A) and survival (panel B). GRFT-treated mice exhibited no weight loss. SARS-CoV-infected mice without GRFT treatment had a 30% survival rate and a ˜25% decrease in weight in those that survived. Results in panels A are presented as means±standard errors. n=7*, P≤0.05 by ANOVA.

FIG. 3 is a chart of relative SARS-CoV-2 RNA abundance over time on MatTek™ human broncho-epithelial airway cultures. The EpiAirway™ tissues were prepared and cultured according to the vendor suggested methods. Tissues were pretreated with Q-GRFT at concentrations ranging from 0.01 to 10 μg/ml prior to challenge with SARS-CoV-2 at MOI of 0.1. Progeny virus release at the apical surface was determined quantitative RT-PCR. Duplicate wells were used for each concentration of Q-GRFT tested.

FIG. 4 depicts images of VERO E6 cells exposed to SARS-CoV-2 after treatment with wild-type griffithsin (WT-GRFT), the Q-griffithsin mutant (Q-GRFT), and a negative control GRFT with binding sites modified to no longer bind sugars (Lec-GRFT) at stated concentrations.

FIG. 5 depicts images of VERO E6 cells exposed to SARS-CoV-2 after treatment with WT-GRFT, Q-GRFT, and Lec-GRFT at stated concentrations. Panels at the bottom show VERO E6 cells incubated with (VC) or without (CC) SARS-CoV-2.

FIG. 6 is a chart depicting the stability of Q-GRFT formulation 1 at a concentration of 7.5 mg/ml at various temperatures.

FIG. 7 is a chart depicting the stability of Q-GRFT formulation 2 at a concentration of 7.5 mg/ml at various temperatures.

FIG. 8 is a chart depicting the stability of Q-GRFT formulation 3 at a concentration of 7.5 mg/ml at various temperatures.

FIG. 9 is a chart depicting the stability of Q-GRFT formulation 4 at a concentration of 1.0 mg/ml at various temperatures.

FIG. 10 is a chart depicting the stability of Q-GRFT formulation 5 at a concentration of 1.0 mg/ml at various temperatures.

FIG. 11 is a chart depicting the stability of Q-GRFT formulation 6 at a concentration of 1.0 mg/ml at various temperatures.

FIG. 12 is a chart depicting the stability of Q-GRFT formulation 30 at a concentration of 7.5 mg/ml at various temperatures.

FIG. 13 is a chart depicting the binding affinity of Q-GRFT with MERS-CoV S protein.

FIG. 14 is a chart depicting the body weight of mice in days after infection by MERS-CoV.

### DETAILED DESCRIPTION OF THE PREFERRED EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described. Wild-type griffithsin is a protein consisting of a single polypeptide, so the terms griffithsin protein and griffithsin polypeptide are used interchangeably.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

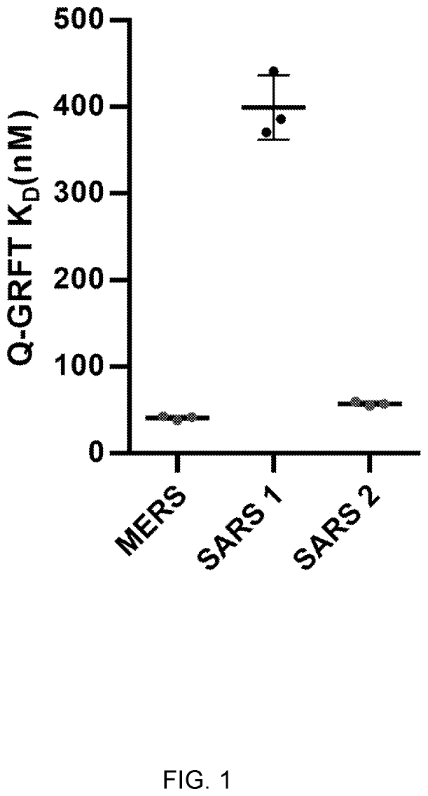

The trimeric envelope spike glycoprotein (S) mediates cellular entry of currently known coronaviruses. SARS-CoV-2 uses the human angiotensinogen-2 (ACE2) molecule as its primary attachment receptor, with cell surface Protease™ PRSS2 mediating S protein priming for entry. The broad spectrum antiviral lectin griffithsin (GRFT) binds oligomannose glycans that represent a significant fraction of the N-linked glycan molecules present on the heavily glycosylated coronavirus S protein. We showed that GRFT binds to, and strongly inhibits viral entry of a broad array of coronaviruses, including SARS-CoV; MERS-CoV; and SARS-CoV-2 (Table 1: FIG. 1).

TABLE 1
Antiviral activity of GRFT against coronaviruses
known to infect humans

| | | | | Selectivity |
|---|---|---|---|---|
| Coronavirus | | EC₅₀ | IC₅₀ | Index |
| Species | Strain | (μg/ml) | (μg/ml) | (SI) |
| SARS-CoV | Urbani | 0.61 | >100 | >164 |
| | Tor-II | 0.61 | >100 | >164 |
| | CuHK | 0.78 | >100 | >128 |
| | Frank | 1.19 | >100 | >83 |
| HCoV | OC43 | 0.16 | 52 | 325 |
| | 229E | 0.18 | >10 | >56 |
| | NL63 | <0.0032 | 10 | >3100 |
| MERS-CoV | EMC/2012 | <0.124 | >2 | >16 |
| SARS-CoV-2 | USA-WA1/2020 | <0.1 | >10 | >100 |

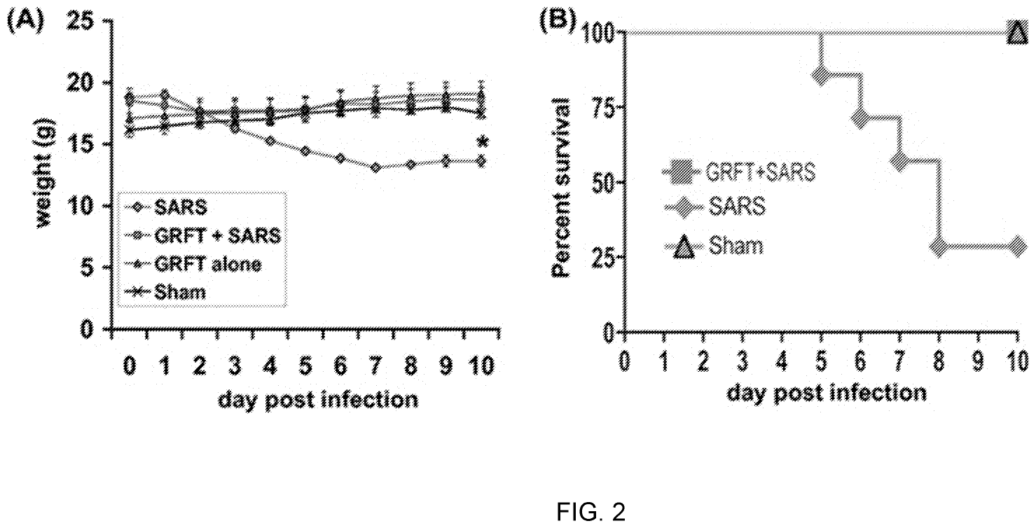

The human ACE2 protein transgenic mouse model was originally developed for use in assessing SARS-CoV pathogenicity. Recently, this model was shown to also support replication of SARS-CoV-2. Infected animals displayed moderate weight loss and clinical disease. The authors detected virus replication in lung. The typical histopathology observed in this model was interstitial pneumonia with infiltration of significant lymphocytes and monocytes in alveolar interstitium, and accumulation of macrophages in alveolar cavities. Viral antigens were observed in the bronchial epithelial cells, alveolar macrophages and alveolar epithelia. Intranasal treatment of mice with GRFT completely protected all animals from challenge with mouse adapted SARS-CoV (FIG. 2). These studies mirror the inventors' unpublished data in the Rhesus macaque model of MERS-CoV, where animals were treated with nebulized GRFT, followed by an aerosol challenge with MERS-CoV. Although some animals showed some evidence of viral infection, viral loads in lungs from GRFT treated animals were about 2 logs lower than in the sham treated group.

Experiments were conducted with multiple GRFT mutants constituting polypeptides with the following amino acid residue sequence: SLTHRKFGGSGGSPFSGLSSIAVRSGSYLDAIIIDGVHHGGSGGNLSPTFTFGSGEYISNX₁T IRSGDYIDNISFX₂TNX₃GRRFGPYGGSGGSANTLSNVKVIQINGX₄X₅GDYLDSLD X₆YYX₇QY (SEQ ID NO: 1), wherein X₁ can be M or V, X₂ is E or Q, X₃ can be M, A, K, V, F, L, I, Q, R, or G, X₄ can be S or R, X₅ can be A or S, X₅ can be I or F, and X₇ can be E or Q. In wild-type GRFT, X₁ is M, X₂ is E, X₃ is M, X₄ is S, X₅ is A, X₆ is I, and X₇ is E (SEQ ID NO: 2).

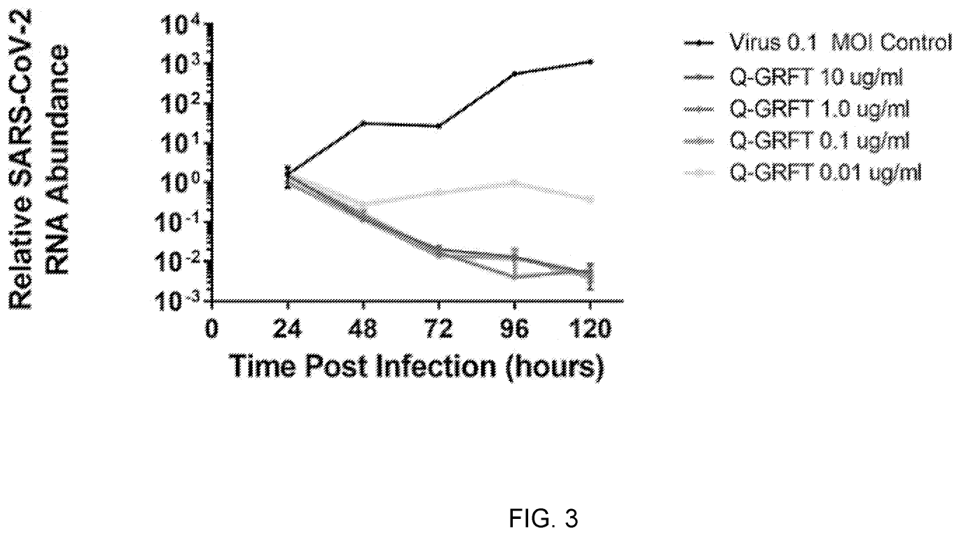

Intranasal treatment with Q-GRFT, an engineered, stability-enhanced version of GRFT, is also protective against transmission of Nipah virus (NiV) in Syrian Golden Hamsters. In Q-GRFT, X₁ is M, X₂ is E, X₃ is Q, X₄ is S, X₅ is A, X₅ is I, and X₇ is E (SEQ ID NO: 18). Other GRFT mutant sequences are shown and discussed in U.S. Publication No. 2002/0087359 and U.S. Pat. No. 10,501,507, both of which are incorporated herein by reference. NiV is a bat-origin, highly pathogenic paramyxovirus that causes frequently fatal encephalitis and respiratory disease in humans. Data on efficacy of GRFT against Coronaviruses SARS-CoV and MERS-CoV, and paramyxovirus NiV demonstrates strong proof of concept that intranasal Q-GRFT treatment could also prevent SARS-CoV-2. This hypothesis is supported by preliminary data showing that pre-exposure treatment with Q-GRFT protected 3-dimensional human airway tissues from SARS-CoV-2 infection, reducing viral replication by over 5 logs in a dose-dependent fashion (FIG. 3).

In some embodiments, the present invention is an intranasal spray for delivery of Q-GRFT into the upper respiratory tract for broad-spectrum Coronavirus pre-exposure prophylaxis. The Q-GRFT nasal spray is a non-vaccine broad spectrum prophylactic that would be particularly suited for individuals who urgently require a product that reduces their risk of upper and lower respiratory tract infection by SARS-CoV-2, such as, for example, front-line healthcare workers, military personnel who must live and work in close quarters, and vulnerable populations—the aged and people with pre-existing morbidities. The COVID-19 pandemic has spread rapidly in advance of effective vaccines or treatments. Development and production of SARS-CoV-2 vaccine is likely at least one year away, and unfortunately the risk of antibody dependent enhancement (ADE) of infection associated with SARS-CoV and some animal coronaviruses necessitates cautious clinical development. If and when a vaccine is available in the future, immunocompromised people may not mount sufficient immune response to provide protection and a Q-GRFT nasal spray would still provide protection in those populations. A Q-GRFT nasal spray may be used to provide a dosage, such as, for example, a daily dosage, effective in inhibiting infection from SARS-CoV-2, endemic coronaviruses, future pandemic coronaviruses, and other viruses and other respiratory pathogens.

The Q-GRFT nasal spray is a topically administered, on-demand product and, unlike a vaccine, does not require a host immune response for protection. Because no immune response is required for activity, the Q-GRFT nasal spray could provide protection for immunocompromised individuals or those who don't adequately mount an immune response. Moreover, topical delivery of this drug eliminates systemic exposure of Q-GRFT, thus reducing the potential for drug-drug interactions and likelihood of systemic side effects as compared to delivery by injection. As an added value, Q-GRFT has broad spectrum coronavirus activity against endemic coronaviruses (four commonly circulating strains infecting humans). In FIG. 3, it is shown that MatTek EpiAirway 3-dimensional bronchial epithelium tissues support replication of SARS-CoV-2.

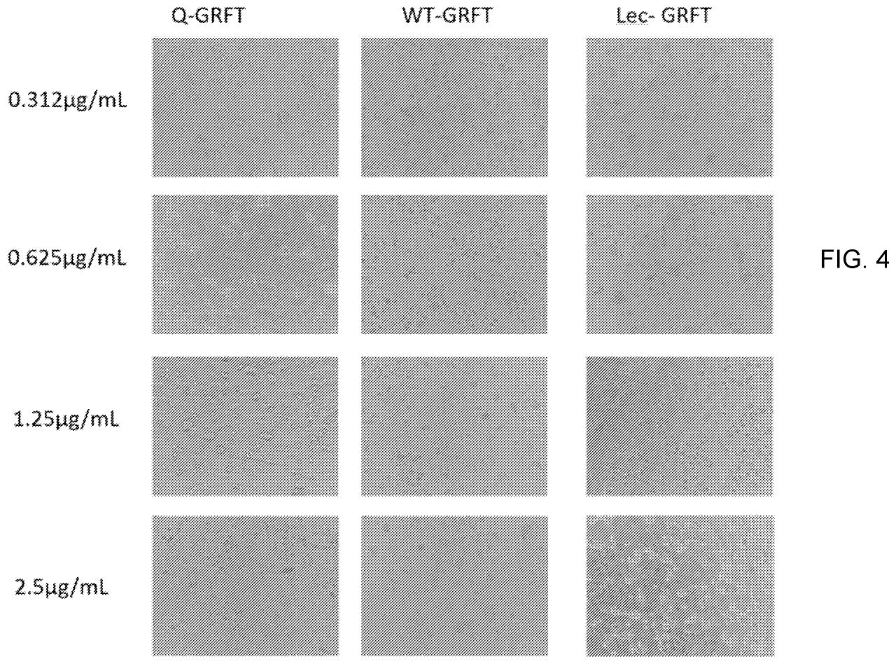

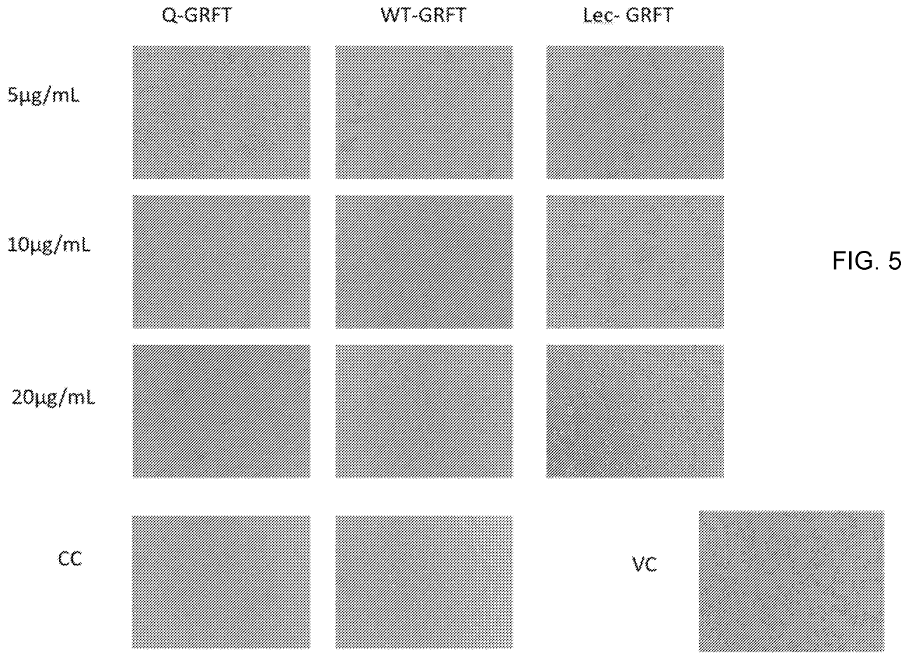

FIGS. 4 and 5 depict a cytopathic effect assay illustrating the anti-SARS-CoV-2 activity of Q-GRFT. The three columns correspond to WT-GRFT, Q-GRFT, and the negative control Lec-GRFT, and the rows correspond to concentrations of the provided proteins. Panels at the bottom on FIG. 5 show VERO E6 cells incubated with (VC) or without (CC) SARS-CoV-2. Infection with SARS-CoV-2 induces cytopathic effects including formation of syncytia (large multinucleated cells caused by virus-induced cell fusion). As shown, the cytopathic effects of SARS-CoV-2 are reduced in the presence of WT-GRFT and Q-GRFT in a dose-dependent manner, as cell treated with WT-GRFT or Q-GRFT appear more similar to the CC uninfected controls.

Q-GRFT in phosphate-buffered saline (PBS) solution compatibility was screened and studied with selected pharmaceutical inactive ingredients. These ingredients included preservatives, viscosity modifiers, and pH modifiers. Mixtures of Q-GRFT at the concentration levels intended to be used clinically (including, but not limited to 10 mg/ml, 7.5 mg/ml and 1.0 mg/ml) with individual excipients and combinations of excipients were made. The samples were packaged, sealed and stored at room temperature and at an accelerated condition of 40° C. and relatively humidity (RH) of 75% and then tested at select time points for parameters including appearance, Q-GRFT drug content, and degradation to evaluate physicochemical stability.

Compatibility with Preservatives

For a preserved nasal spray formulation development, the selection of preservative system is important as it plays a major role in determining the product shelf life and safety. Screened preservatives included imidurea, methylparaben, propylparaben, chlorobutanol, potassium sorbate, sorbic acid, citric acid, acetic acid, benzalkonium chloride (BKC), benzyl alcohol and phenylethanol. The mixture of Q-GRFT with individual or combination of preservatives were pH adjusted to their corresponding effective pH range and monitored for Q-GRFT physicochemical chemical stability. Results showed that Q-GRFT was compatible with methylparaben and propylparaben, and their salt forms with increased aqueous solubility are optimal for formulation development which involved no heat process. Potassium sorbate and imidurea were viable for short term use but ineffective on providing longer shelf life as compared to the formulations comprising parabens which make them less favorable. Q-GRFT was not compatible with the most commonly used BKC resulting in immediate precipitation upon mixing. Antimicrobial effectiveness per USP <51> Antimicrobial Effectiveness Test was performed to confirm effectiveness for the selected preservative systems.

Compatibility with Viscosity Modifier

Viscosity modifiers not only change product deposition in the nasal cavity but also increase product local residence time to provide improved product efficacy.

A range of viscosity modifiers were studied for their ability to increase product viscosity and compatibility with Q-GRFT drug substance in PBS. These viscosity modifiers include water soluble cellulose (e.g., variable grades of hydroxylpropyl methylcellulose (HPMC) and hydroxyethyl cellulose (HEC)), dispersible cellulose (e.g. different composition of microcrystalline cellulose/sodium carboxymethylcellulose (MCC)), polyvinylpyrilidone (PVP) gums and polysaccharides (e.g. iota, lambda and kappa carrageenans). In addition, poloxamers, nonionic copolymers of polyethylene and polyoxypropylene was also screened for their unique thermoreversible properties. Results showed that Q-GRFT was physicochemically compatible with water soluble cellulose (HPMC and HEC) resulting in stable clear transparent solutions. It was also compatible with Lambda carrageenan. Q-GRFT was found not to be compatible with MCC and other types of carrageenans with observed phase separation either upon contact with excipients or upon storage.

Compatibility with pH Modifier

Strong acid (hydrochloride, HCL) and weak organic acids (citric acid and acetic acid) were used for pH adjustment. It was found that HCL was compatible with Q-GRFT when used alone and in combination with other excipients including polymers and preservatives which made it an optimal selection for formulation development. Citric acid and acetic acid were both compatible with Q-GRFT when used alone and in combination with some excipients, however, citric acid when combined with certain preservatives, e.g. methylparaben and propylparaben, resulted in crystal like precipitation associated with loss of Q-GRFT when product was stored for approximately 2 months.

Formulation Process (HPMC and HEC)

Production of Griffithsin-based nasal spray formulations includes, in some embodiments, the steps of (1) weighing Q-GRFT API (in PBS solution) in a container; (2) weighing polymer (HPMC or HEC) and add into Q-GRFT solution little by little while stirring to dissolve the polymer while avoiding lump formation; (3) weighing and adding other excipients (parabens, maltitol, xylitol, etc.), stirring to dissolve the excipients; (4) adjusting pH to target (4.5 or 6.5) using HCl; and (5) calculate remaining amount of solvent (PBS or MilliQ (i.e., purified water)), and quantum satis to volume (“QS,” meaning keep adding until the desired volume is reached). In step 5, depending on the composition of the nasal spray formulation, either PBS or MilliQ water were used to adjust the final osmolality to be isotonic or near isotonic.

Formulation Process (Carrageenan)

Production of Griffithsin-based nasal spray formulations includes, in some embodiments, the steps of (1) weighing Q-GRFT API (in PBS solution) in a container; (2) weighing and adding preservative, stirring to dissolve; (3) weighing and adding other excipients (parabens, maltitol, etc.), stirring to dissolve the excipients; (4) adjusting pH to target (4.5 or 6.5) using HCl; (5) adding carrageenan stock solution per formula amount (see following tables) to the container, and continue stirring to substantial uniformity; (6) adjusting pH to target (4.5 or 6.5) if needed; and (7) calculate remaining amount of solvent (PBS or MilliQ), and QS to volume. The carrageenan stock solution added in step 5 is prepared in some embodiments at 1.5% w/w by (i) weighing purified water in a container arranging for automated stirring of the water; (ii) heating the purified water to about 80° C. if preparing iota carrageenan or omitting this heating step if preparing lambda carrageenan; (iii) weighing carrageenan powder and slowly adding it to the stirring water to avoid lump formation; (iv) continued stirring until dissolved; and (v) discontinuing application of heat, if iota carrageenan was used.

It should be understood that the aforementioned production processes are exemplary processes for small scale production, and that different processes may be used in large scale production of Griffithsin-based nasal spray formulations.

In some embodiments, the Q-GRFT nasal spray was developed according to the target profile recited in Table 2. The following Tables 3-6 recite the compositions of Q-GRFT-containing formulations and Tables 7-9 recite characterizations of the formulations. Individual formulations are referenced by their code numbers recited in each table.

TABLE 2
Target profile for Q-GRFT nasal spray

| | |
|---|---|
| Test Parameter | Tentative specification |

Physicochemical characteristics

| | |
|---|---|
| Identification | RT comparable to API (reference |
| | standard) |
| Assay (n = 3) | 90-110% |
| Assay for preservative (n = 3) | 80-120% |
| pH | 4.5-7.4 |
| Osmolality (n = 3) | Isotonic-near isotonic |
| Viscosity | 15 cps |
| Appearance | Clear solution, free of visible |
| | particles |
| Package integrity | Visual observation |
| Leachable/Extractable | TBD |

Microbial testing

| | |
|---|---|
| Preservative effectiveness testing | USP <51> |
| Microbial limits testing | USP <61> <62> |

Spray characteristics

| | |
|---|---|
| Droplet size distribution | 50-100 μm |
| Spray pattern | Ellipsoid of uniform density |
| | Ovality: 1-1.3 |
| Spray content uniformity (n = 10) | 80-120% of label claim |
| Plume geometry | Plume angle: >45° |
| | Plume width: >25 mm |
| Priming and repriming | To characterize |

TABLE 3
Q-GRFT HPMC based formulation composition

| | | | Ingredients (% w/w) | | | | | | | | |
|---|---|---|---|---|---|---|---|---|---|---|---|
| | | | | | | Q-GRFT | | | | | |
| Q-GRFT | | | Methylparaben | Propylparaben | Potassium | in PBS | | | | | PBS or |
| Concentration | Code | Lot Number | Sodium | Sodium | Sorbate | (11.8 mg/ml) | HEC | Maltitol | Xylitol | HCL 2N | MilliQ |
| 10 mg/ml | 10#1 | LW15P59 | 0.18 | 0.02 | — | 84.75 | 0.20 | — | — | QS to pH 6.5 | QS to 100 |
| | 10#2 | LW15P58 | — | — | 0.1 | 84.75 | 0.20 | — | — | QS to pH 4.5 | QS to 100 |
| | 10#3 | LW15P60 | — | — | 0.2 | 84.75 | 0.20 | — | — | QS to pH 4.5 | QS to 100 |
| 7.5 mg/ml | 1 | LW15P96 | 0.18 | 0.02 | — | 63.56 | 0.20 | — | — | QS to pH 6.5 | QS to 100 |
| | 2 | LW15P97 | 0.18 | 0.02 | — | 63.56 | 0.20 | 1.00 | — | QS to pH 6.5 | QS to 100 |
| | 7 | LW15P108 | 0.18 | 0.02 | — | 63.56 | 0.20 | — | 1.00 | QS to pH 6.5 | QS to 100 |
| | 10 | LW15P112 | 0.18 | 0.00 | — | 63.56 | 0.20 | — | — | QS to pH 6.5 | QS to 100 |
| 1.0 mg/ml | 4 | LW15P99 | — | — | 0.2 | 8.47 | 0.20 | — | — | QS to pH 4.5 | QS to 100 |
| | 5 | LW15P100 | 0.18 | 0.02 | — | 8.47 | 0.20 | 1.00 | — | QS to pH 6.5 | QS to 100 |
| | 8 | LW15P109 | 0.18 | 0.02 | — | 8.47 | 0.20 | — | 1.00 | QS to pH 6.5 | QS to 100 |

TABLE 4
Q-GRFT HEC based formulation composition

| | | | Ingredients (% w/w) | | | | | | | | |
|---|---|---|---|---|---|---|---|---|---|---|---|
| | | | | | | Q-GRFT | | | | | |
| Q-GRFT | | | Methylparaben | Propylparaben | Potassium | in PBS | | | | | PBS or |
| Concentration | Code | Lot Number | Sodium | Sodium | Sorbate | (11.8 mg/ml) | HEC | Maltitol | Xylitol | HCL 2N | MilliQ |
| 10 mg/ml | 10#4 | LW15P61 | 0.18 | 0.02 | — | 84.75 | 0.20 | — | — | QS to pH 6.5 | QS to 100 |
| | 10#5 | LW15P63 | — | — | 0.1 | 84.75 | 0.20 | — | — | QS to pH 4.5 | QS to 100 |
| 7.5 mg/ml | 3 | LW15P98 | 0.18 | 0.02 | — | 63.56 | 0.20 | 1 | — | QS to pH 6.5 | QS to 100 |
| | 12 | LW16P126 | 0.18 | 0.02 | — | 63.56 | 0.20 | — | — | QS to pH 6.5 | QS to 100 |
| 1.0 mg/ml | 6 | LW1SP101 | 0.18 | 0.02 | — | 8.47 | 0.20 | 1 | — | QS to pH 6.5 | QS to 100 |
| | 9 | LW15P110 | 0.18 | 0.02 | — | 8.47 | 0.20 | — | 1 | QS to pH 6.5 | QS to 100 |
| | 11 | LW15P125 | — | — | 0.2 | 8.47 | 0.20 | — | — | QS to pH 4.5 | QS to 100 |

TABLE 5
Q-GRFT carrageenan (Lambda) based formulation composition

| | | | Ingredients (% w/w) | | | | | | | |
|---|---|---|---|---|---|---|---|---|---|---|
| | | | | | | Q-GRFT | | | | |
| Q-GRFT | | | Methylparaben | Propylparaben | Potassium | in PBS | | | | PBS of |
| Concentration | Code | Lot Number | Sodium | Sodium | Sorbate | (11.8 mg/ml) | Lambda | Maltitol | HCL 4N | MilliQ |
| 7.5 mg/ml | 14 | LW15P134 | 0.18 | 0.02 | — | 63.56 | 0.2 | — | QS to pH 6.5 | QS to 100 |
| | 18 | LW15P143_1 | — | — | 0.2 | 63.56 | 0.2 | 1 | QS to pH 4.5 | QS to 100 |
| | 21 | LW16P145 | — | — | 0.2 | 63.56 | 0.2 | 1 | QS to pH 4.5 | QS to 100 |
| | 20 | LW15P144 | 0.18 | 0.02 | — | 63.56 | 0.2 | 1 | QS to pH 6.5 | QS to 100 |
| | 30 | LW15P183 | 0.18 | 0.02 | — | 63.56 | 0.05 | 1 | QS to pH 6.5 | QS to 100 |
| 1.0 mg/ml | 26 | LW15P150 | — | — | 0.2 | 10.53 | 0.2 | — | QS to pH 4.5 | QS to 100 |
| | 27 | LW15P151 | 0.18 | 0.02 | — | 10.53 | 0.2 | — | QS to pH 6.5 | QS to 100 |

TABLE 6
Q-GRFT carrageenan (Iota) based formulation composition

| | | | Ingredients (% w/w) | | | | | | | |
|---|---|---|---|---|---|---|---|---|---|---|
| | | | | | | Q-GRFT | | | | |
| Q-GRFT | | | Methylparaben | Propylparaben | Potassium | in PBS | | | | PBS or |
| Concentration | Code | Lot Number | Sodium | Sodium | Sorbate | (11.8 mg/ml) | Lambda | Maltitol | HCL 4N | MilliQ |
| 7.5 mg/ml | 13 | LW15P133 | 0.18 | 0.02 | — | 63.56 | 0.2 | — | QS to pH 6.5 | QS to 100 |
| | 15 | LW15P135 | 0.18 | 0.02 | — | 63.56 | 0.2 | 1 | QS to pH 6.5 | QS to 100 |
| | 23 | LW15P147 | 0.18 | 0.02 | — | 63.56 | 0.2 | 1 | QS to pH 6.5 | QS to 100 |
| 1.0 mg/ml | 16 | LW15P136 | — | — | 0.2 | 10.53 | 0.2 | — | QS to pH 4.5 | QS to 100 |
| | 24 | LW15P148 | — | — | 0.2 | 10.53 | 0.2 | — | QS to pH 4.5 | QS to 100 |
| | 25 | LW15P149 | 0.18 | 0.02 | — | 10.53 | 0.2 | — | QS to pH 6.5 | QS to 100 |

TABLE 7
HPMC based formulation characterization

| | | | | osmo- | | |
|---|---|---|---|---|---|---|
| Q-GRFT | | | | lality | Vis- | |
| Concen- | | Lot | | (m0sm/ | cosity | |
| tration | Code | Number | pH | kg) | (cps) | Appearance |
| 10 mg/ml | 10#1 | LW15P59 | 6.6 | 310 | 4-10 | Transparent |
| | | | | | | clear solution |
| | 10#2 | LW15P58 | 5 | 358 | 4-10 | Transparent |
| | | | | | | clear solution |
| | 10#3 | LW15P60 | 4.5 | 330 | 4-10 | Transparent |
| | | | | | | clear solution |
| 7.5 mg/ml | 1 | LW15P96 | 6.5 | 269 | 4-10 | Transparent |
| | | | | | | clear solution |
| | 2 | LW15P97 | 6.5 | 289 | 4-10 | Transparent |
| | | | | | | clear solution |
| | 7 | LW15P108 | 6.5 | 369 | 4-10 | Transparent |
| | | | | | | clear solution |
| | 10 | LW15P112 | 6.5 | 318 | 4-10 | Transparent |
| | | | | | | clear solution |
| 1.0 mg/ml | 4 | LW15P99 | 4.5 | 313 | 4-10 | Transparent |
| | | | | | | clear solution |
| | 5 | LW15P100 | 6.5 | 283 | 4-10 | Transparent |
| | | | | | | clear solution |
| | 8 | LW15P109 | 6.5 | 306 | 4-10 | Transparent |
| | | | | | | clear solution |

TABLE 8
HEC based formulation characterization

| | | | | osmo- | | |
|---|---|---|---|---|---|---|
| Q-GRFT | | | | lality | Vis- | |
| Concen- | | Lot | | (m0sm/ | cosity | |
| tration | Code | Number | pH | kg) | (cps) | Appearance |
| 10 mg/ml | 10#4 | LW15P61 | 6.5 | 302 | 4-10 | Transparent |
| | | | | | | clear solution |
| | 10#5 | LW15P63 | 5.28 | 296 | 4-10 | Transparent |
| | | | | | | clear solution |
| 7.5 mg/ml | 3 | LW15P98 | 6.53 | 284 | 4-10 | Transparent |
| | | | | | | clear solution |
| | 12 | LW15P126 | 6.53 | 304 | 4-10 | Transparent |
| | | | | | | clear solution |
| 1.0 mg/ml | 6 | LW15P101 | 6.53 | 309 | 4-10 | Transparent |
| | | | | | | clear solution |
| | 9 | LW15P110 | 6.49 | 311 | 4-10 | Transparent |
| | | | | | | clear solution |
| | 11 | LW15P125 | 4.5 | 266 | 4-10 | Transparent |
| | | | | | | clear solution |

TABLE 9
Carrageenan based formulation characterization

| | | | | osmo- | | |
|---|---|---|---|---|---|---|
| Q-GRFT | | | | lality | Vis- | |
| Concen- | | Lot | | (m0sm/ | cosity | |
| tration | Code | Number | pH | kg) | (cps) | Appearance |
| 7.5 mg/ml | 20 | LW15P144 | 6.48 | 290 | 2-10 | Transparent |
| | | | | | | clear solution |
| 1.0 mg/ml | 27 | LW15P151 | 6.5 | 270 | 2-10 | Transparent |
| | | | | | | clear solution |
| 7.5 mg/ml | 30 | LW15P183 | 6.5 | 316 | 1-10 | Slightly |
| | | | | | | translucent |
| | | | | | | solution |
| 7.5 mg/ml | 23 | LW15P147 | 6.52 | 305 | 2-10 | Slightly |
| | | | | | | translucent |
| | | | | | | solution |
| 1.0 mg/ml | 25 | LW15P149 | 6.54 | 271 | 2-10 | Slightly |
| | | | | | | translucent |
| | | | | | | solution |

Formulation Stability

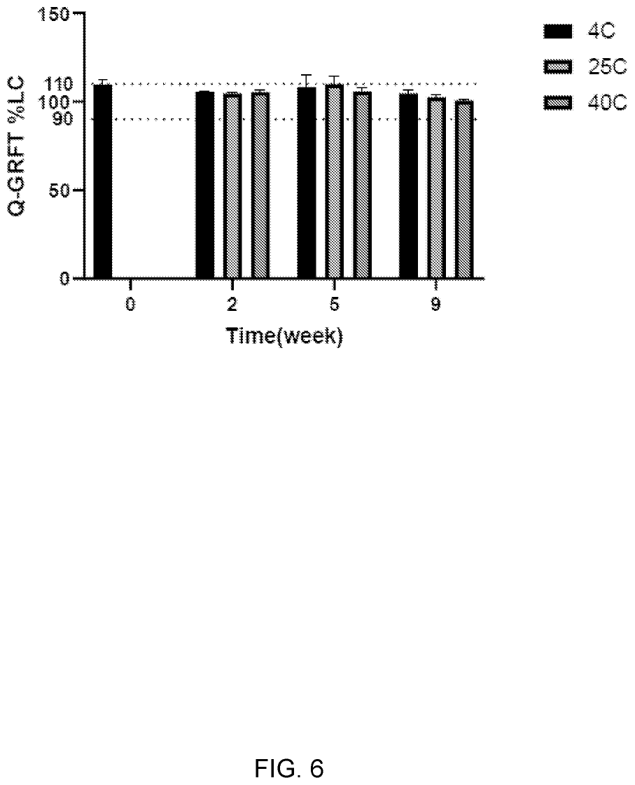

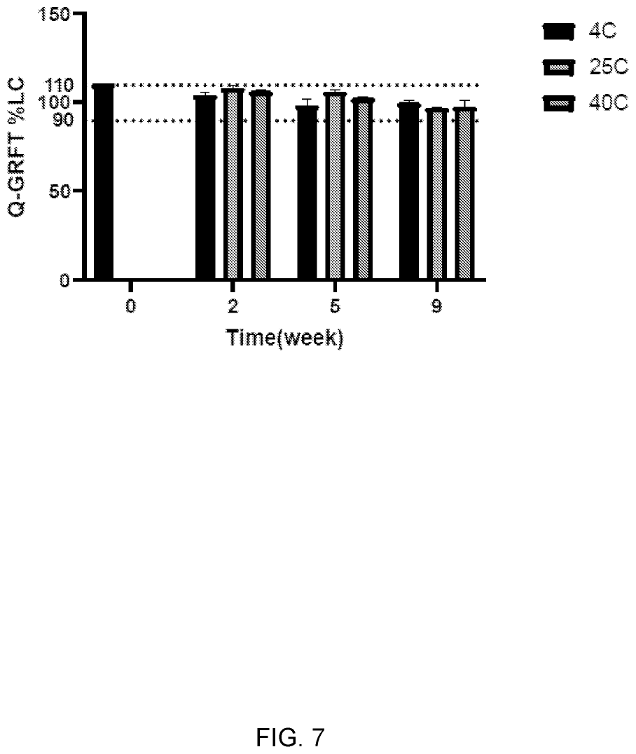

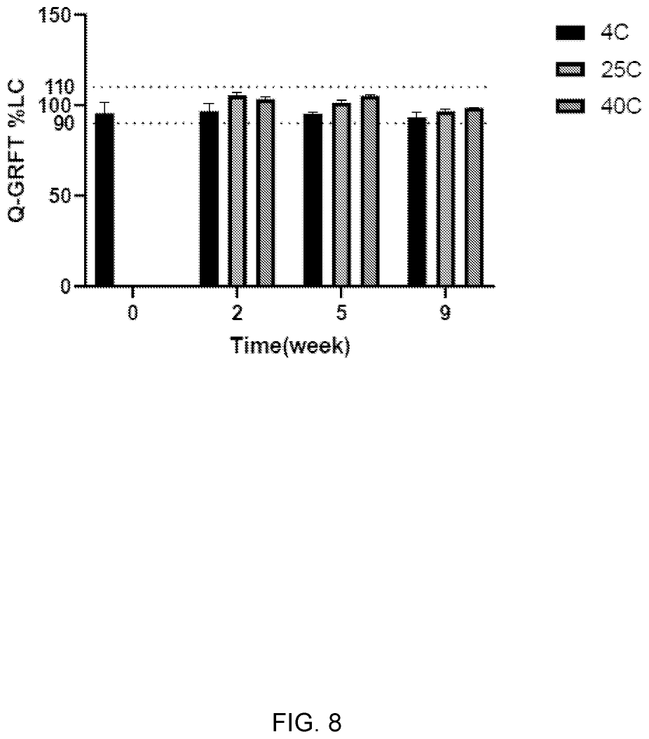

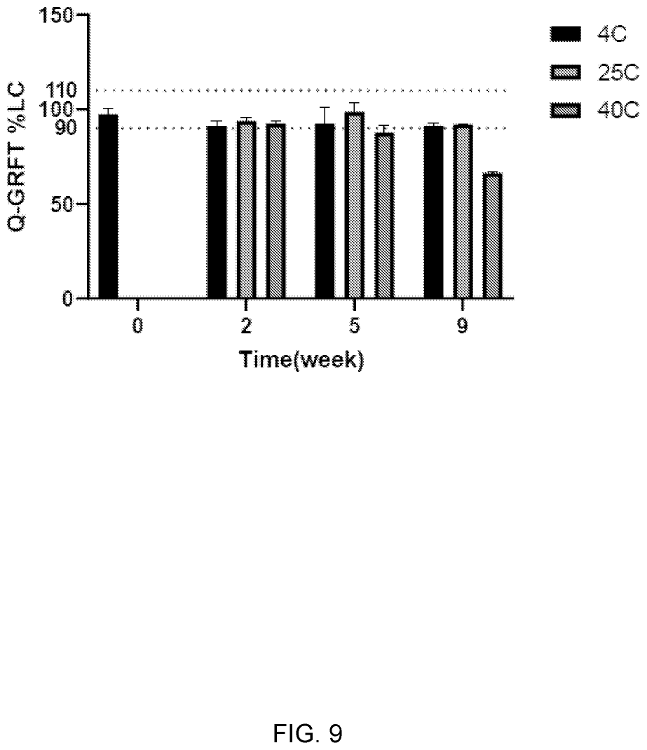

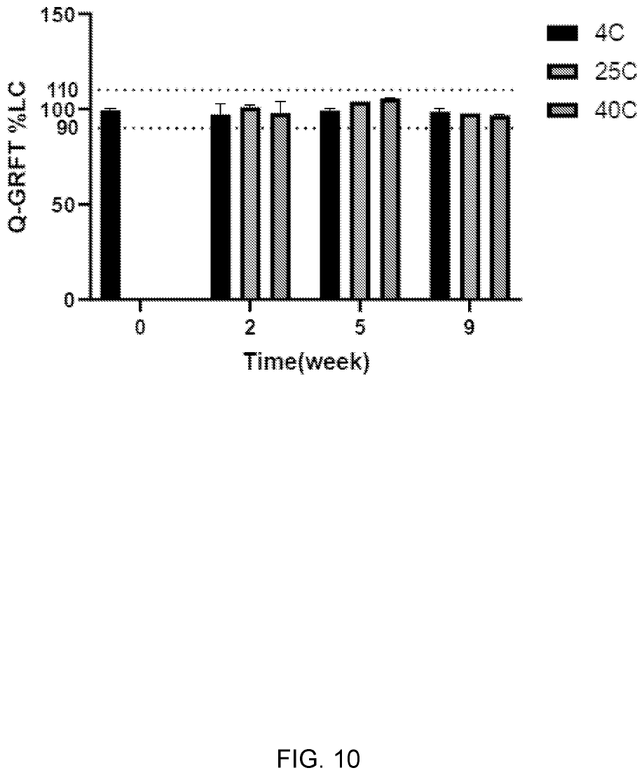

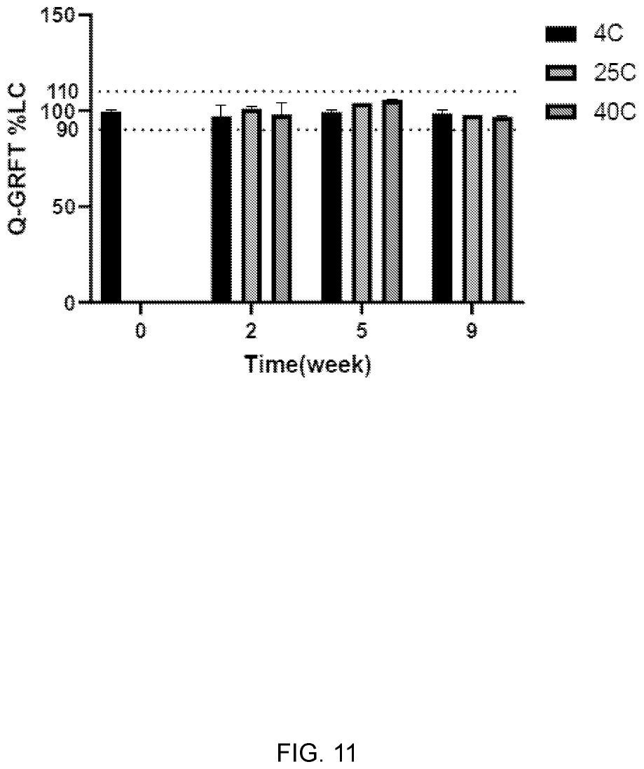

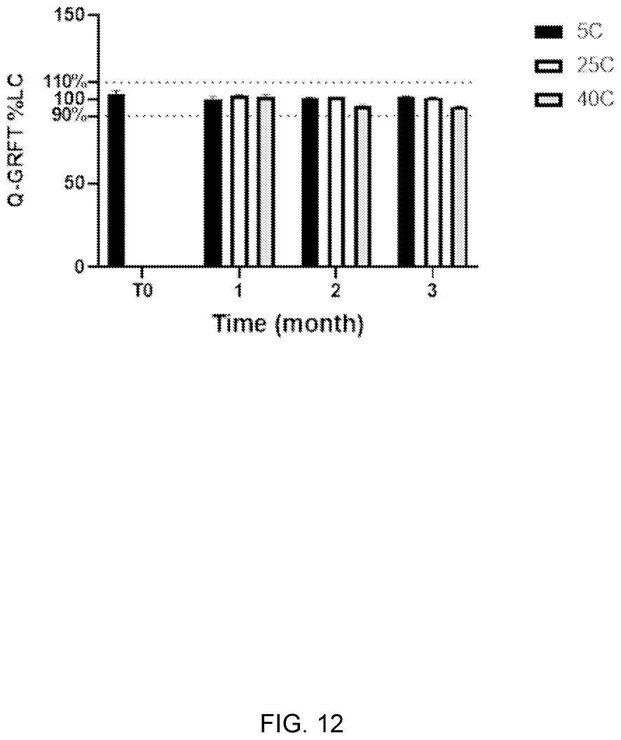

Selected formulations have been prepared, packaged into glass scintillation vials with polyseal caps and put on stability at three conditions (4° C. or 5° C., 25° C./RH60% and 40° C./RH75%). Samples are tested at each timepoint and evaluated for stability including appearance, pH, viscosity, drug content and degradation. Results for formulations 1-6 and 30 are depicted in FIGS. 6-12, respectively. The y-axis measurement of “% LC” refers to % label claim, i.e., the detected concentration as compared to the target concentration, that being 7.5 mg/ml for formulations 1-3 and 30, and 1.0 mg/ml for formulations 4-6. In addition, no significant pH and osmolality changes were observed. With respect to formulation 30 specifically, the Q-GRFT content remained within specification (90%-110% of label claim (LC), LC=7.5 mg/mL). pH ranged from 6.4-6.6 and osmolarities ranged from 312-320 mOsm/kg.

Formulation Evaluation: Cell Based and EpiAiway Tissue Toxicity

Formulations were screened for toxicity in both a cell based and EpiAirway™ constructed tissue model. These studies showed that all formulations tested had no significant toxicity as compared to commercially marketed nasal product controls. The cell based model was also applied for excipient screening evaluations.

A panel of formulations are being tested in two tissue efficacy models including EpiAiway™ and EpiNasal™ tissues. In this study, the tissues were exposed to high SARS-CoV-2 virus at level of MOI 0.1, and treated with either Q-GRFT drug substance and formulation on a daily bases. TCI D50 was measured. QGRFT API was shown to be effective by viral load reduction of 3-4 logs as compared to the virus control groups in both models, and formulation 3 (composition shown in Table 4 and characterization shown in Table 8) was shown to be effective in both models.

MERS-CoV S Protein

Middle East Respiratory Syndrome Coronavirus (MERS-CoV) is a virus first reported in 2012. MERS-CoV causes MERS, a viral respiratory illness characterized by fever, cough, shortness of breath, often followed by more severe complications, such as pneumonia and kidney failure. MERS-CoV S protein is a surface spike protein on the MERS-CoV viral shell which facilitates MERS-CoV entry into host cells.

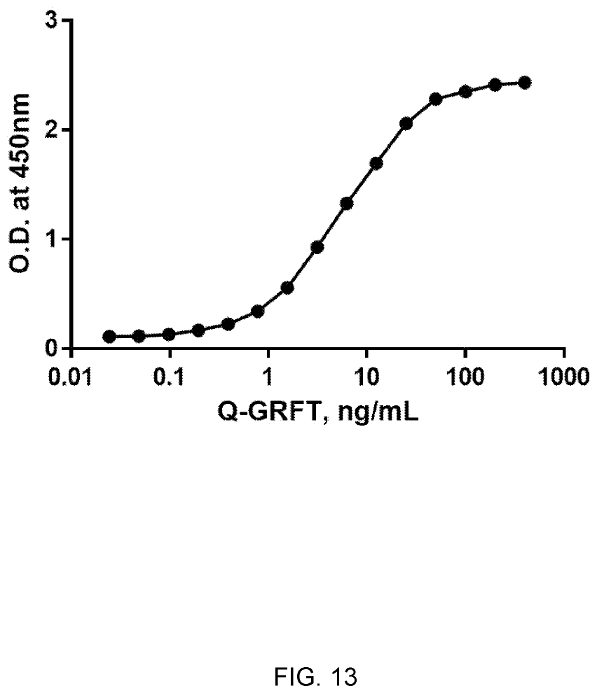

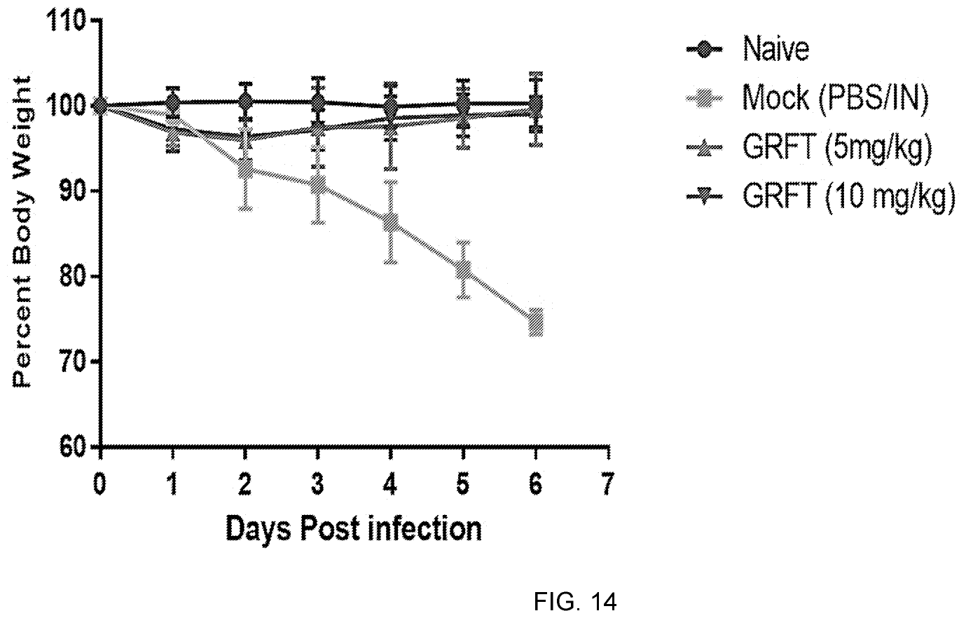

FIG. 13 depicts the binding affinity of Q-GRFT to MERS-CoV S protein. FIG. 14 depicts the body weight of mice after infection with MERS-CoV. Naive (i.e., uninfected) mice had no significant change in body weight in the six days after infection. Mice treated with Q-GRFT (5 mg/kg body weight or 10 mg/kg body weight) experienced a minor loss of body weight but recovered to normal. In contrast, mice receiving a mock treatment of PBS experienced a significant decrease in body weight and died six days after infection. This data indicates that Q-GRFT is effective in binding and reducing the negative effects of MERS-CoV.

Q-GRFT has been identified to have activity against a range of viruses thus the nasal spray would have potential as a preventative or therapeutic for any which are transmitted through the upper respiratory tract. In particular, the product has potential as a prophylactic agent against SARS-CoV-2.

Various aspects of different embodiments of the present disclosure are expressed in paragraphs X1, X2, and X3 as follows:

X1. One embodiment of the present disclosure is a method of prophylactically or therapeutically inhibiting an viral infection in a host comprising administering to the host a polypeptide comprising the amino acid sequence of SLTHRKFGGSGGSPFSGLSSIAVRSGSYLDAIIIDGVHHGGSGGNLSPTFTFGSGEYISNX₁T IRSGDYIDNISFX₂TNX₃GRRFGPYGGSGGSANTLSNVKVIQINGX₄X₅GDYLDSLD X₆YYX₇QY, wherein X₁ can be M or V, X₂ can be E or Q, X₃ can be M, A, K, V, F, L, I, Q, R, or G, X₄ can be S or R, X₅ can be A or S, X₆ can be I or F, and X₇ can be E or Q, such that the viral infection is inhibited.

X2. Another embodiment of the present disclosure is an intranasal spray formulation comprising a griffithsin protein in a composition including a compatible preservative and a compatible viscosity modifier.

X3. A further embodiment of the present disclosure is a method of treating or preventing infection with a coronavirus in a patient comprising intranasally delivering the intranasal spray formulation to the patient in a dosage regimen effective to prevent or treat a coronavirus infection, the intranasal spray formulation comprising a griffithsin protein in a composition including a compatible preservative and a compatible viscosity modifier.

Yet other embodiments include the features described in any of the previous statements X1, X2, or X3, as combined with one or more of the following features:

Wherein X₃ is Q.

Wherein X₁ is M, X₂ is E, X₃ is Q, X₄ is S, X₅ is A, X₅ is I, and X₇ is E.

Wherein the viral infection is a coronavirus infection.

Wherein the viral infection is SARS-CoV.

Wherein the viral infection is SARS-CoV-2.

Wherein the viral infection is MERS-CoV.

Wherein the polypeptide is administered to the upper respiratory tract of the host.

Wherein the polypeptide is administered in aerosol form.

Wherein the polypeptide is administered in the form of an intranasal spray.

Wherein the composition is one of the formulations listed in Tables 3, 4, 5, or 6.

Wherein the composition is one of formulations 1, 2, 3, 4, 5, 6, or 30.

Wherein the intranasal spray includes a compatible preservative and a compatible viscosity modifier.

Wherein the compatible preservative is methylparaben or propylparaben.

Wherein the compatible preservative is methylparaben and propylparaben.

Wherein the viscosity modifier is hydroxypropyl methylcellulose, hydroxyethyl cellulose, or lambda carageenan.

Wherein the viscosity modifier is a water-soluble cellulose.

Wherein the viscosity modifier is hydroxyethyl cellulose

Wherein the composition comprises from 0.1 mg/mL to 20 mg/mL, or from 1 mg/mL to 10 mg/mL, or about 7.5 mg/mL of the griffithsin protein.

Wherein the griffithsin protein is Q-Griffithsin.

Wherein the griffithsin protein comprises the amino acid sequence of SLTHRKFGGSGGSPFSGLSSIAVRSGSYLDAIIIDGVHHGGSGGNLSPTFTFGSGEYISNX₁T IRSGDYIDNISFX₂TNX₃GRRFGPYGGSGGSANTLSNVKVIQINGX₄X₅GDYLDSLD X₆YYX₇QY, wherein X₁ can be M or V, X₂ can be E or Q, X₃ can be M, A, K, V, F, L, I, Q, R, or G, X₄ can be S or R, X₅ can be A or S, X₆ can be I or F, and X₇.

Wherein HCl, acetic acid, or citric acid is used to adjust the pH of the composition.

Wherein the pH is adjusted to below 7.

Wherein the pH is adjusted to the range of 4.5 to 6.6.

Wherein the pH is adjusted to about 6.5.

Wherein the composition is contained within a nasal spray device.

Wherein the composition is contained within an aerosol sprayer along with a propellant.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1) A method of prophylactically or therapeutically inhibiting an viral infection in a host comprising administering to the host a polypeptide comprising the amino acid sequence of SLTHRKFGGSGGSPFSGLSSIAVRSGSYLDAIIIDGVHHGGSGGNLSPTFTFGSGEYISNX₁T IRSGDYIDNISFX₂TNX₃GRRFGPYGGSGGSANTLSNVKVIQINGX₄X₅GDYLDSLD X₆YYX₇QY, wherein X₁ can be M or V, X₂ can be E or Q, X₃ can be M, A, K, V, F, L, I, Q, R, or G, X₄ can be S or R, X₅ can be A or S, X₆ can be I or F, and X₇ can be E or Q, such that the viral infection is inhibited.

2) The method of claim 1, wherein X₃ is Q.

3) The method of claim 1, wherein the viral infection is a coronavirus infection.

4) The method of claim 3, wherein the viral infection is SARS-CoV-2.

5) The method of claim 3, wherein the viral infection is MERS-CoV.

6) The method of claim 1, wherein the polypeptide is administered to the upper respiratory tract of the host.

7) The method of claim 1, wherein the polypeptide is administered in aerosol form.

8) The method of claim 1, wherein the polypeptide is administered in the form of an intranasal spray.

9) The method of claim 8, wherein the intranasal spray includes a compatible preservative and a compatible viscosity modifier.

10) The method of claim 9, wherein the compatible preservative is methylparaben or propylparaben.

11) The method of claim 9, wherein the viscosity modifier is hydroxypropyl methylcellulose, hydroxyethyl cellulose, or lambda carageenan.

12) An intranasal spray formulation comprising a griffithsin protein in a composition including a compatible preservative and a compatible viscosity modifier.

13) The intranasal spray formulation of claim 12, wherein the compatible preservative is methylparaben or propylparaben.

14) The intranasal spray formulation of claim 12, wherein the viscosity modifier is hydroxypropyl methylcellulose, hydroxyethyl cellulose, or lambda carageenan.

15) The intranasal spray formulation of claim 12, wherein the composition comprises from 0.1 mg/mL to 20 mg/mL, or from 1 mg/mL to 10 mg/mL, or about 7.5 mg/mL of the griffithsin protein.

16) The intranasal spray formulation of claim 12, wherein the griffithsin protein is Q-Griffithsin.

17) The intranasal spray formulation of claim 12, wherein the griffithsin protein comprises the amino acid sequence of SLTHRKFGGSGGSPFSGLSSIAVRSGSYLDAIIIDGVHHGGSGGNLSPTFTFGSGEYISNX₁T IRSGDYIDNISFX₂TNX₃GRRFGPYGGSGGSANTLSNVKVIQINGX₄X₅GDYLDSLD X₆YYX₇QY, wherein X₁ can be M or V, X₂ can be E or Q, X₃ can be M, A, K, V, F, L, I, Q, R, or G, X₄ can be S or R, X₅ can be A or S, X₆ can be I or F, and X₇.

18) The intranasal spray formulation of claim 17, wherein X₃ is Q.

19) The intranasal spray formulation of claim 12 wherein HCl, acetic acid, or citric acid is used to adjust the pH of the composition.

20) The intranasal spray formulation of claim 12, wherein the composition is contained within a nasal spray device.

21) A method of treating or preventing infection with a coronavirus in a patient comprising intranasally delivering the intranasal spray formulation of claim 12 to the patient in a dosage regimen effective to prevent or treat a coronavirus infection.
